(19) United States
(12) Patent Application Publication
(10) Pub. No.: US 2012/0040655 A1
(43) Pub. Date: Feb. 16, 2012

(54) Cell Phone Based Tampon Monitoring System

(76) Inventor: Kevin B. Larkin, Pebble Beach, CA (US)

(21) Appl. No.: 12/857,470

(22) Filed: Aug. 16, 2010

(51) Int. Cl. H04M 3/00; A61F 13/42; H04M 1/00; A61F 13/20

(52) U.S. Cl. 455/418; 604/385.17; 604/361; 455/556.1

(57) ABSTRACT

A cell phone based tampon monitoring system features a tampon with a sensor in it, a sensor hub in a wired signal connection with the sensor and in a wireless signal connection with a cell phone. Software installed on the cell phone initially identifies the sensor hub upon connection of a fresh tampon, communicates with the sensor hub via a secondary wireless port of the cell phone, processes the received signals, and utilizes the cell phone's user interfaces to privately inform the tampon user and/or telephone forward logged sensor data to a remote third party device. By utilizing conventional cell phones, women may simply and privately monitor their currently inserted tampon and get timely forecasts and alerts. Through ongoing use, a wealth of information about menstruation cycle and other related health issues may be utilized by women and/or their doctors without having to carry along any extra device.

### CROSS REFERENCE

The present application cross references application Ser. No. 12/749,645, filed 30-MAR-2010, titled “Tampon Saturation Monitoring System”, Attorney Docket No. WES1153CIP, of the same inventor Kevin B. Larkin which is herewith incorporated by reference.

### FIELD OF INVENTION

The present invention relates to cellular phone based systems for monitoring sensor signals of sensors in vaginally inserted tampons.

### BACKGROUND OF INVENTION

Users of tampons with sensors need a monitoring device that is readily accessible and conveniently carried along throughout the day. Such a monitoring device is needed to for a private status reporting of the tampon's condition and other communication capabilities that are specific to the nature of a tampon's use and its eventual blood saturation progress. The present invention addresses this need.

A woman's overall health condition may be closely linked to her menstruation cycle. Cycle duration, cycle regularity, cycle intensity and blood conductivity related to iron levels may be some parameters to determine a woman's overall health. Therefore, there exists a need for a convenient way of logging the menstruation cycles on an ongoing base in a time stamped manner to process information for example about cycle durations, cycle regularity, cycle intensity, blood conductivity and fertility periods. Such health information may be telephone forwarded to remote third party devices for example of a doctor. The present invention addresses also this need.

Cell phones are commonly carried in close proximity throughout the day. They also provide a number of features such as distinctive ring, vibration, secondary wireless communication with other than telephoning devices, and custom software execution. All this makes a cellular phone highly suitable as a personal monitoring device in particular in combination with tampons having a sensor such as a blood saturation monitor. Therefore, there exists a need for a system utilizing a cell phone as a tampon saturation monitoring device. The present invention addresses also this need.

### SUMMARY OF INVENTION

A cell phone based tampon monitoring system features a tampon with a sensor in it, a sensor hub in a wired signal connection with the sensor and in a wireless signal connection with a cell phone. Commercially available cell phones may be adapted by installing a software application on it. The software may feature a wireless identification routine for initially identifying a sensor hub, a signal communication routine that is communicating with the sensor hub via a secondary wireless port that is common in cell phones at the time of this invention, a signal processing algorithm, and an information routine that is accessing and utilizing the cell phone's user interfaces and/or telephoning feature to forward the logged data to a remote third party device. By utilizing conventional cell phones and without having to carry along any extra device, women may be given a simple and private means to monitor the saturation of their currently inserted tampon, get forecasts and alerts in a timely manner and through ongoing use get a wealth of information about her menstruation cycle and other related health information that may be utilized by her and/or her doctor.

### BRIEF DESCRIPTION OF THE FIGURES

FIG. 1 is a schematic of a cell phone based tampon monitoring system.

FIG. 2 is a schematic of a cell phone with an installed tampon monitoring software.

FIGS. 3A-3C are time graphs of various tampon sensor signals received by the cell phone from the sensor hub.

FIG. 4 is a schematic transparent perspective depiction of a tampon with a tampon sensor.

### DETAILED DESCRIPTION

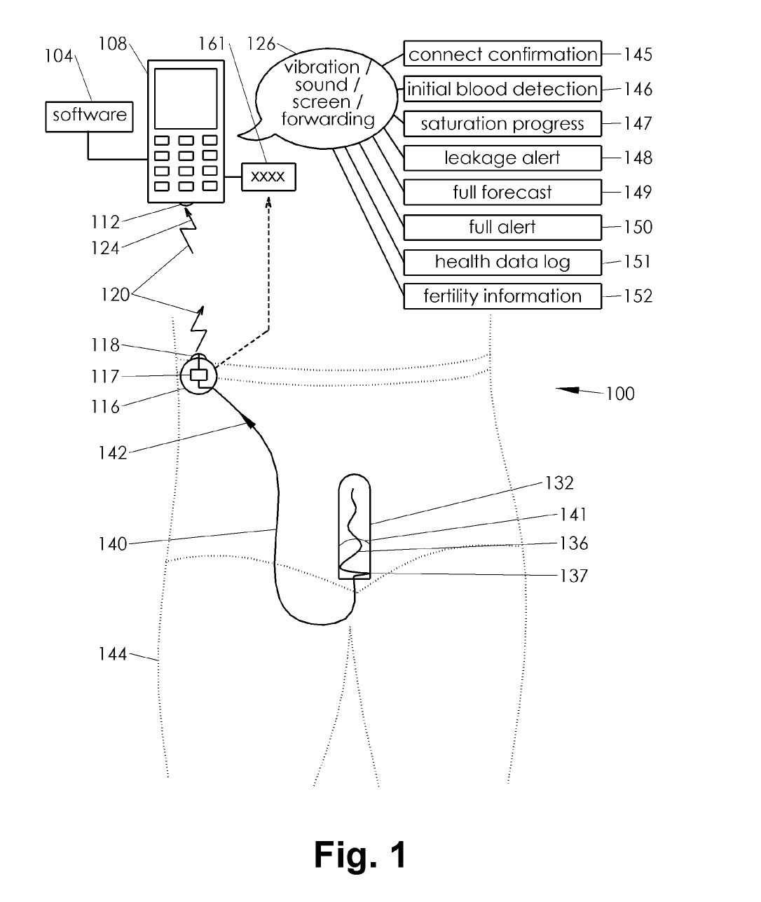

Referring to FIG. 1, a cell phone based tampon monitoring system 100 includes a sensor 136 in a tampon 132 worn by a female user 144, a sensor hub 116 in a wired signal connection 140 with the sensor 136, and a personal communication device 108 in a wireless signal connection 120 with the sensor hub 116. The personal communication device 108 such as preferably a cell phone or other well known hand held or pocket size electronic communication device receives via the wireless signal connection 140 a wireless tampon signal 124 that is generated by the sensor hub 116 in conjunction with a tampon signal 142 generated by the sensor 136 and received by the sensor hub 116 via the wired connection 140. The cell phone 108 processes the wireless tampon signal 124 preferably by means of an installed software application 104 into a monitoring information 126 that is presented as at least one of a sensor hub connect information 145, an initial blood detection information 146, a tampon saturation progress information 147, a tampon leakage alert 148, a tampon full forecast 149, a tampon full alert 150, a health data log 151 and a fertility information 152. The wired signal connection 140 may be a dual wire electric cable or optical cable in between the sensor 136 and the sensor hub 116 as described in the cross referenced applications. There the sensor hub 116 is described as a notifier or buzzer that is removable attached to the user's 144 undergarment. The wireless signal connection 120 includes a hub transceiver or sender 118 and a cell phone transceiver 112 that preferably operate according to the well known Bluetooth™ technology. The wired tampon signal 142 may be converted by a hub processor 117 of the sensor hub 116 into the wireless signal 124 and may be transmitted in a predetermined periodic fashion to minimize consumption of battery power in the sensor hub 116 as may be clear to anyone skilled in the art. The hub processor 117 may uniquely encrypt the wireless tampon signal, meaning that no other sensor hub 116 may have the same wireless tampon signal. The cell phone 108 may decrypt the wireless tampon signal upon entering a decryption code 161 that is uniquely associated with the sensor hub 116. This provides for a safe one directional wireless communication between sensor hub 116 and cell phone 108 and consequently for a most simplistic configuration of the sensor hub 116 as may be well appreciated by anyone skilled in the art.

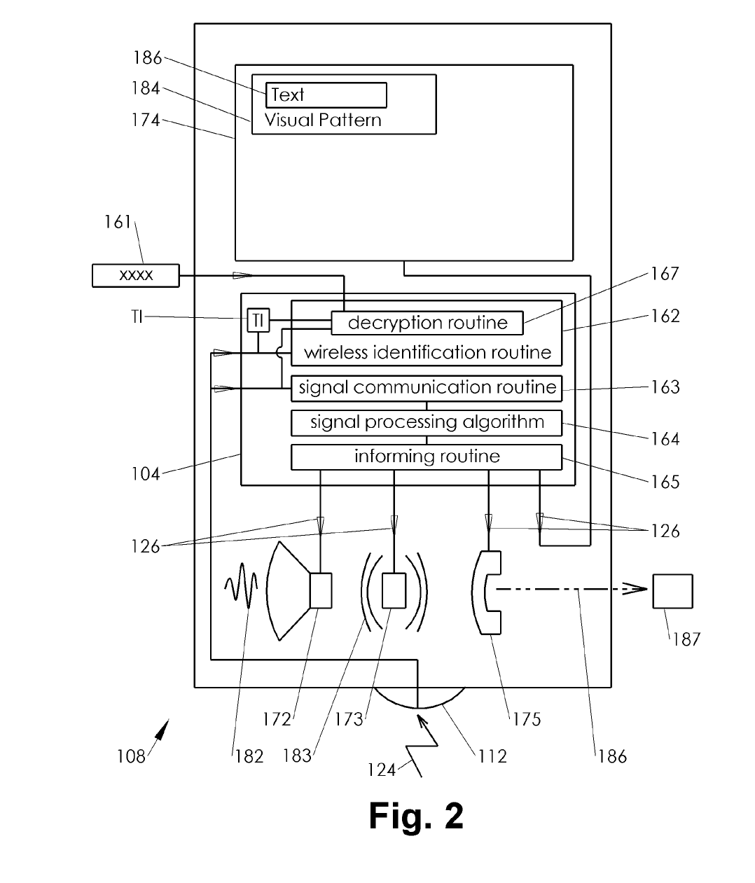

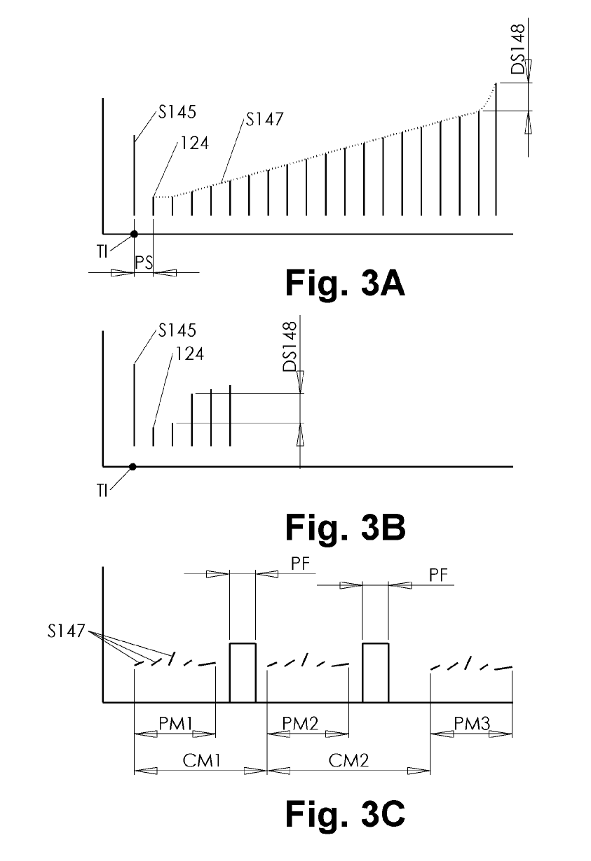

Referring to FIG. 2, the tampon monitoring software application 104 may feature a wireless identification routine 162, a signal communication routine, a signal processing algorithm 164 and an information routine 165. The wireless identification routine 162 is identifying via the wireless signal communication port 112 the tampon sensor hub 116 and is generating an initial time stamp TI of an identification moment with the tampon sensor hub 116. The identification moment may be that moment where a newly inserted tampon 136 is connected to the sensor hub 116 and the sensor hub 116 transmits a connect confirmation signal S145 as shown in FIGS. 3A-3C. In case of encrypted wireless tampon signals 124, a decryption routine 167 may be part of the wireless identification routine that is decrypting the encrypted wireless tampon signal 124 in accordance with the user 144 entered decryption code 161. That way and once a new sensor hub 116 is put into service for the first time, the identification moment may also be the moment, the decryption code 161 is entered. Once wireless connection 120 is established and the sensor hub 116 is identified, the signal communication routine 163 is communicating via the wireless communication port 112 with the sensor hub 116 and is receiving preferably periodically a wireless tampon signal 124. The signal processing algorithm 164 is processing from the wireless tampon signal 124 and the initial time stamp TI the monitoring information 126. Other data obtained with the cell phone's 108 features such as GPS and the like, may also be incorporated for example as activity or location information into the monitoring information 126. This may be helpful for example in correlating fluctuations in long term menstrual cycle data or other long term health data with particular life events and the like. The information routine 165 is accessing and utilizing at least one of the user interface 172, 173, 174 and a telephoning feature 175 of the cell phone 108 to communicate the monitoring information 126.

The signal processing algorithm 164 is processing the wireless tampon signal 124 into at least one but preferably all of the sensor hub connect confirmation 145, the initial sensor blood detection information 146, the tampon saturation progress 147, the tampon leakage alert 148, the tampon full forecast 149, the tampon full alert 150, the health data log 151, and the fertility information 152. The information routine may 165 may be accessing and utilizing a well known telephoning feature 175 of the personal communication device 108 in case of which the monitoring information 126 is a telephone forwarded information 186 such as a well known digital data stream to a remote third party device 187. The telephoning feature 175 may rely on a telephone network and/or a well known Wifi™ network. The telephone forwarded information 186 may be transmitted over a telephone connection and/or an internet connection. Alternately or additionally, the information routine 165 may be accessing a user interface 172, 173, 174 of the personal communication device 108. The accessed and utilized user interface 173 may be a well known vibrator 173 in case of which the monitoring information is provided as a distinctive vibration pattern 183. That way, the user 144 may be most discretely informed. The accessed and utilized user interface 173 may be a well known sound device 172 such as a speaker in case of which the monitoring information is provided as a distinctive sound pattern 182. The accessed and utilized user interface 173 may be a well known screen 174 in case of which the monitoring information is provided as a distinctive visual pattern 184. The visual pattern 184 may be a blinking screen, abstract color patterns, graphic or pictorial content or any combination thereof. The distinctive visual pattern 184 may be also a text 186. The tampon monitoring software application 104 may be provided as a data package available via an internet connection and/or a telephone connection of the personal communication device 108.

Referring to FIGS. 3A, 3B, 3C, a new Tampon 132 may be vaginally inserted by a user 144 and the wired connection 142 established with tampon sensor hub 116, while the software application 104 has readied the cell phone 108 to receive the wireless tampon signal 124. In case also a new tampon sensor hub 116 is utilized, a corresponding decryption code 161 is entered by the user 144 into the cell phone 108. As soon as the wired connection 140 is established, the hub processor 117 sends out a connect confirmation signal S145 that receives the initial time stamp TI preferably in the cell phone 108. Nevertheless, the scope of the invention includes also embodiments in which time stamping is provided by the hub processor 117. The processing algorithm 164 may issue the connect confirmation 145 such that the user 144 knows that the tampon monitoring system 100 is properly working.

Following the connect confirmation signal S145, at preferably periodic intervals PS are sent by the sensor hub 116 a number of wireless tampon signals 124. Sending at periodic intervals PS preserves battery power of the sensor hub 116 as may be well appreciated by anyone skilled in the art. The cell phone 108 may be put into receive mode at those periodic intervals PS. Once the first wireless tampon signal 124 is received, the initial blood detection message 146 may be issued. A number of wireless tampon signals 124 may be processed by the application software 104 preferably into a saturation progression curve S147 and the corresponding saturation progress information 147 may be issued. From the steepness of the saturation progression curve S147 and/or gain between consecutive wireless tampon signals 124, the processing algorithm 164 may process also the tampon full forecast 149.

As shown in FIG. 1, the tampon sensor 136 may feature at the peripheral end of the tampon 132 a leakage and full detection loop 137 that is in close proximity to the tampon's 132 circumference and that has a distinctive signal signature DS148 such that a tampon leakage along the tampon circumference is detected distinctively from a gradual blood saturation progression 141. The distinctive signal signature DS148 may be a distinctive conductivity gain in the case of the sensor 136 being a electrically resistive sensor 132 as described in the cross referenced applications and as may be clear to anyone skilled in the art. When the tampon 132 becomes full, the distinctive leakage signal DS148 occurs at the end of a gradual increase between consecutive wireless tampon signals 124 and the tampon full alert 150 is issued. In case of tampon leakage, the distinctive signal signature DS148 occurs at an early stage as shown in FIG. 3B. The processing algorithm 164 may interpret from the previous low number of wireless tampon signals 124 and/or the short time span between initial time stamp TI the tampon leakage and issue the leakage alert 148.

As shown in FIG. 3C, a number of saturation progression curves S147 during menstrual periods PM1, PM2, PM3 may be logged by the processing algorithm 164 together with menstrual cycle periods CM1, CM2 between the menstrual periods PM1, PM2, PM3. In addition, amount of menstrual blood as well as blood iron levels from correlated blood conductivity may be processed from the absolute levels of the saturation progression curves S147 as may be well appreciated by anyone skilled in the art. From that, the health data log 151 may be generated by the processing algorithm 164 as well as fertility information 152 including fertility periods PF as may be clear to anyone skilled in the art. In times in between the menstruation periods PM1, PM2, PM3, the sensor 132 be operated as temperature sensor in case of which the tampon monitoring system 100 may be utilized to automatically detect vaginal temperature changes correlated to the fertility period as may be clear to anyone skilled in the art.

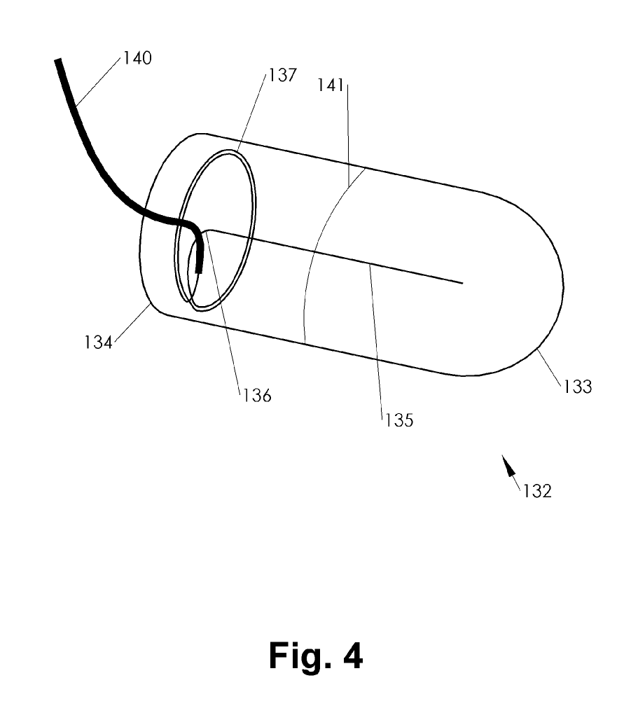

Utilizing the leakage and full detection loop 137 with its distinctive leakage or full signal DS148 provides for a reliable identification of tampon full saturation and tampon leakage irrespective varying blood conductivity and varying absolute levels of the wireless tampons signals 124. Referring to FIG. 4, the tampon sensor 136 preferably has a string like structural composition, which makes it highly suitable to integrate it within and combine it with the rolled gauze material tampons 132 are commonly made of. Such string like sensor 136 may have a substantially continuous line sensitivity as described in the cross referenced application. To provide the distinctive leakage or full signal DS148 with such a string like sensor 136, it may be assembled within the tampon 132 with a substantially directly between peripheral tampon end 134 and tampon insertion end 133 extending sensor portion 135 that provides a gradual and signal 142 in correspondence with the progressing blood saturation boundary 141. But once the blood saturation boundary 141 reaches the leakage and full detection loop 137, a substantial sensor length gets simultaneously wetted, which results in the distinctive leakage or full signal DS148. Likewise in the case of tampon leakage where blood runs along the circumference of the tampon 132, a substantial length of sensor string 136 gets simultaneously wetted along the leakage and full detection loop 137 that is in immediate proximity to the tampon's 132 circumference. In both cases and irrespective varying blood conductivity, tampon 132 leakage as well as its upcoming full saturation are reliably detected.

The scope of the invention described in the figures and specification above is set forth by the following claims and their legal equivalent:

What is claimed is:

1. The cell phone based tampon monitoring system comprising:
a. a sensor in a tampon;
b. a sensor hub in a wired signal connection with said sensor;
c. a cell phone in a wireless signal connection with said sensor hub across which said cell phone is receiving a wireless tampon signal that is generated by said sensor hub in conjunction with a wired tampon signal generated from said sensor and received by said sensor hub via said wired signal connection;
wherein said cell phone processes said wireless tampon signal into a monitoring information that is presented as at least one of a sensor hub connect confirmation, an initial blood detection information, a tampon saturation progress information, a tampon leakage alert, a tampon full forecast, a tampon full alert, a health data log and a fertility information.

2. The cell phone based tampon monitoring system of claim 1, wherein wireless tampon signal is initially time stamped.

3. The cell phone based tampon monitoring system of claim 2, wherein said initial time stamping is provided in said cell phone.

4. The cell phone based tampon monitoring system of claim 1, wherein said sensor hub further comprises a hub processor that is converting said wired tampon signal said wireless tampon signal in a predetermined periodic fashion.

5. The cell phone based tampon monitoring system of claim 1, further comprising a decryption code that is uniquely associated with said sensor hub, and wherein said sensor hub further comprises a hub processor that is uniquely encrypting said wireless tampon signal and said cell phone is decrypting said wireless tampon signal upon entering said decryption code.

6. The cell phone based tampon monitoring system of claim 1, wherein said sensor further comprises at a peripheral end of said tampon a leakage detection loop that is in close proximity to a circumference of said tampon and that has a distinctive signal signature such that a tampon leakage along said tampon circumference is detected distinctively from a gradual blood saturation progression along said tampon.

7. The cell phone based tampon monitoring system of claim 1, wherein said sensor is a temperature sensor.

8. A tampon monitoring software application for a personal communication device a comprising:
a. a wireless identification routine that is identifying via a wireless signal communication port of said personal communication device a tampon sensor hub and that is generating an initial time stamp of an identification moment with said tampon sensor hub;
b. a signal communication routine that is communicating with an identified one of said tampon sensor hub via said wireless communication port and that is receiving a wireless tampon signal from said tampon sensor hub;
c. a signal processing algorithm that is processing at least from said wireless tampon signal and said initial time stamp a monitoring information; and
d. an information routine that is accessing and utilizing at least one of user interface and a telephoning feature of said personal communication device to communicate said monitoring information.

9. The tampon monitoring software application of claim 8, wherein said wireless identification routine further comprises a decryption routine that is decrypting an encrypted one of said wireless tampon signal in accordance with a user entered decryption code.

10. The tampon monitoring software application of claim 8, wherein said signal processing algorithm is processing said wireless tampon signal into at least one of a sensor hub connect confirmation, an initial sensor blood detection, a tampon saturation progress, a tampon leakage alert, a tampon full forecast, a tampon full alert, a health data log, and a fertility information.

11. The tampon monitoring software of claim 8, wherein said information routine is accessing and utilizing a telephoning feature of said personal communication device and wherein said monitoring information is a telephone forwarded information to a remote third party device.

12. The tampon monitoring software application of claim 8, wherein said information routine is accessing and utilizing a user interface of said personal communication device.

13. The tampon monitoring software application of claim 12, wherein said user interface is a vibrator and wherein said monitoring information is provided as a distinctive vibration pattern.

14. The tampon monitoring software application of claim 12, wherein said user interface is a sound device and wherein said monitoring information is provided as a distinctive sound pattern.

15. The tampon monitoring software application of claim 12, wherein said user interface is a screen and wherein said monitoring information is a distinctive visual pattern.

16. The tampon monitoring software application of claim 15, wherein said distinctive visual pattern is a text.

17. The tampon monitoring software application of claim 8, wherein said personal communication device is a cell phone.

18. The tampon monitoring software application of claim 8 being provided as a data package available via an internet connection.

19. The tampon monitoring software application of claim 8, being provided as a data package available via a telephone connection of said cell personal communication device.

20. A cell phone comprising:
a. a wireless signal communication port that is in wireless communication with a tampon sensor hub providing a wireless tampon signal;
b. a tampon monitoring software application that is time stamping and processing said wireless tampon signal into a monitoring information;
c. a user interface that is communicating said monitoring information via at least one of a vibrator, a sound device, a screen of said cell phone and a telephoning feature to a remote third party device.
